(19) United States
(12) Patent Application Publication
(10) Pub. No.: US 2026/0158185 A1
(43) Pub. Date: Jun. 11, 2026

(54) REMOVABLE ASSEMBLY FOR A DIFFUSING APPARATUS, AND DIFFUSING APPARATUS

(71) Applicant: CAELIMP, LABEGE (FR)

(72) Inventors: Arnaud JOURDAN, TOULOUSE (FR); Samuel DUFOUR, TOULOUSE (FR); Philippe RIVIERE, TOULOUSE (FR)

(21) Appl. No.: 18/704,342

(22) PCT Filed: Oct. 26, 2022
(86) PCT No.: PCT/EP2022/079910

§ 371 Date: Apr. 24, 2024

(30) Foreign Application Priority Data
Oct. 27, 2021 (FR) FR2111442

(51) Int. Cl. A61L 9/03; A01M 1/20

(52) U.S. Cl. CPC A61L 9/037; A01M 1/2072; A01M 1/2077; A61L 2209/133

(57) ABSTRACT

The invention relates to a removable assembly (50; 250; 350; 450) for a diffusing apparatus, the removable assembly including:
a storage container (60; 260; 360; 460) including a drain orifice (61; 261; 361; 461), and
a dispensing member positioned at the outlet of the drain orifice and connected to the drain orifice, the dispensing member including a hollow body (70; 270; 370; 470) having an evaporation surface situated on the exterior of the storage container.

The storage container (60; 260; 360; 460) has a concave exterior shape surrounding at least partially a central free space (62; 262; 362; 462) so as to uncover a contact surface of the porous body on the side opposite the evaporation surface, the contact surface being accessible through the central free space (62; 262; 362; 462).

The invention further relates to a diffusing apparatus (10; 710; 810; 910; 1910; 2010) adapted to receive the removable assembly in a housing (31).

### TECHNICAL FIELD

The invention relates to the field of diffusing apparatus intended to disperse into the air in the vapor state a substance in the liquid or solid state at ambient temperature. The invention relates more particularly to a removable assembly that can be used in such diffusing apparatus.

### Prior Art

Diffusing apparatus of the aforementioned type is known for example from the document WO 2019/243734 A1. In that apparatus a storage container containing the substance is connected to a dispensing member. The dispensing member includes microchannels forming an outlet arranged in the channel in order to constitute therein a substance evaporation zone. A heating member is arranged on or in the dispensing member in such a manner as to control a flow of the substance through the dispensing member.

### SUMMARY OF THE INVENTION

One idea behind the invention consists in proposing an arrangement of the dispensing member and of the storage container that facilitates placement of the heating member relative to the dispensing member.

The invention therefore proposes a removable assembly for a diffusing apparatus, the diffusing apparatus being intended to disperse into the air in the vapor state a substance in the liquid or solid state at ambient temperature, the removable assembly comprising:
a storage container for containing the substance and including a drain orifice, the drain orifice being oriented downwards when the removable assembly is in a position of use; and
a dispensing member positioned at the outlet of the drain orifice and connected to the drain orifice, the dispensing member including a porous body having an evaporation surface situated at the exterior of the storage container to evaporate the substance into the surrounding air,
in which the storage container has a concave exterior shape surrounding at least partially a central free space so as to uncover a contact surface on the porous body on the side opposite the evaporation surface, the contact surface being accessible via the central free space.

The heating member enables control of the flow of the substance through the porous body simply by heating or not heating the porous body. The physical principles on which this control of the flow of the substance through the porous body is based are described in the document WO 2019/243734 A1 already cited above or in the document WO 2020/254733 A1.

Thanks to the fact that the storage container has a concave exterior shape at least partially surrounding a central free space in such a manner as to uncover a contact surface of the porous body, the heating member can be placed on the contact surface of the dispensing member simply by passing it through the central free space of the storage container. This can in particular be achieved by arranging the heating member in a housing of the apparatus that is intended to receive the removable assembly. It is then particularly simple to position the heating member relative to the dispensing member.

Embodiments of such a removable assembly may have one or more of the following features.

In accordance with one embodiment the porous body has a porosity in an interior part of the porous body that is lower than a porosity in an exterior part of the porous body surrounding the interior part.

In accordance with one embodiment the porous body includes a wick made of wood, textile, ceramic, polymer or porous metal material obtained by sintering a metal powder or a metal alloy powder.

In accordance with one embodiment the porous body includes a void situated in line with the central free space surrounded by the storage container and the contact surface is an interior surface of the void.

The heating member is therefore received in part in the void in the porous body so as to come into contact with the contact surface. A result of this is that the contact between the heating member and the porous body is effected over a larger surface than by planar contact. This tends to facilitate controlling the temperature of the porous body and thereby controlling the flow of the substance through the porous body.

The void is preferably blind. Alternatively, the void is not blind, that is to say it opens through the porous body.

In accordance with one embodiment the porous body includes a cylindrical portion.

In accordance with one embodiment the central free space extends in a direction coaxial with the cylindrical portion of the porous body.

In accordance with one embodiment the void extends in an axial direction of the cylindrical portion of the porous body.

In accordance with one embodiment the porous body further includes a projecting portion of hemispherical or spherical dome shape, the projecting portion being disposed at an axial end of the cylindrical portion opposite the central free space.

In accordance with one embodiment the void extends into the projecting portion.

Various exterior shapes of the storage container are possible.

In accordance with one embodiment the storage container extends along a main axis.

In accordance with one embodiment an axis of the cylindrical portion of the porous body coincides with the main axis.

In accordance with one embodiment the direction in which the central free space extends is parallel to the main axis.

In accordance with one embodiment the axial direction in which the void extends is parallel to the main axis.

In accordance with one embodiment the porous body exhibits symmetry of revolution about the main axis.

In accordance with one embodiment in the position of use the main axis is parallel to the direction of acceleration due to gravity.

In accordance with one embodiment the storage container includes a lower wall, the lower wall being at a lower end of the storage container along the main axis.

In accordance with one embodiment the drain orifice is in the lower wall.

In accordance with one embodiment the lower wall includes a spot facing, the central free space opening into the spot facing and the drain orifice being formed in the spot facing.

In accordance with one embodiment said concave exterior shape of the storage container is annular. For example, a section of the storage container perpendicular to the main axis is of annular shape.

In accordance with one embodiment the storage container exhibits symmetry of revolution about the main axis.

In accordance with one embodiment said concave exterior shape of the storage container is the shape of a “C”. For example, a section of the storage container perpendicular to the main axis is the shape of a “C”.

In accordance with one embodiment the drain orifice is blocked by a foil.

In accordance with one embodiment the removable assembly further includes a perforator device disposed between the porous body and the foil, the perforator device including a plurality of teeth and/or needles configured to perforate the foil when the porous body is moved in the direction of the foil.

In accordance with one embodiment the removable assembly further includes a wick support element configured to be assembled to the storage container, the wick support element including a support portion, the support portion being configured to support the porous body and including a through-hole through which the porous body passes and the support portion including a plurality of teeth and/or needles configured to perforate the foil when the wick support is assembled to the storage container.

In accordance with one embodiment the wick support element is configured to be assembled with the storage container by screwing it thereto. In this case the plurality of teeth and/or needles perforate the foil when the wick support element is screwed onto the storage container. For example, the wick support element may include an assembly portion including an interior thread configured to cooperate with an exterior thread on the storage container.

In accordance with one embodiment the wick support element is configured to be assembled with the storage container by screwing it thereto in an intermediate position in which the plurality of teeth and/or needles do not perforate the foil and in a final position in which the plurality of teeth and/or needles do perforate the foil, the passage from the intermediate position to the final position necessitating a greater tightening force than the tightening force necessary to screw the wick support element as far as the intermediate position. For example, the assembly portion may include an interior projection configured to cooperate with exterior projections on the storage container, the passage from the intermediate position to the final position necessitating a tightening force sufficient for the interior projection to be able to be forced past the exterior projections.

In accordance with one embodiment said substance includes at least one compound selected from semiochemical molecules, pheromones, allomones, kairomones, synomones of natural or synthetic origin.

In accordance with one embodiment the substance is a solution containing at least one sexual or non-sexual pheromone, an allomone, a synomone or a kairomone intended to cause a positive or negative response in the target species, the behavioral result of which may be sexual confusion, confusion of another kind, sexual attraction, attraction of another kind, repulsion of any kind, in arthropods, including arachnids, or hexapods, including in particular insects, including harmful insects.

In accordance with one embodiment the substance is a solution containing at least one pheromone or sexual pheromone, an allomone, a synomone or a kairomone intended to provoke a positive or negative result in the target species, the behavioral result of which may be in particular pacification, relaxation, euphoria or intimidation in mammals and birds.

In accordance with one embodiment the substance contains a solvent chosen from isopropyl myristate, glycol dipropylene, monomethyl glycol dipropylene ether, and an isoparaffin hydrocarbon, for example an L or P or N or V isoparaffin.

In accordance with one embodiment the substance includes at least one compound in the group formed by odoriferous agents usable on man or animals, semiochemical substances, cosmetic agents, essential oils, perfumes, disinfecting agents and phytosanitary and agricultural agents. In accordance with one embodiment the substance is a solution containing at least one compound from the above group.

In accordance with one embodiment the substance contains at least one compound in the group formed by odoriferous agents usable on man, cosmetic agents, essential oils, perfumes, disinfecting agents. In accordance with one embodiment the substance is a solution containing at least one compound from the above group.

In accordance with one embodiment the odiferous agents usable on animals are chosen from fatty acids or the esterified form of said fatty acids such as methyl oleate, methyl palmitate, dimethyl azelate and dimethyl pimelate.

In accordance with one embodiment the substance has in the liquid state a viscosity greater than 1 cPa·s at 25° C., for example greater than 8 cPa·s at 25° C., and less than 1 cPa·s at 60° C.

In accordance with one embodiment the substance has at atmospheric pressure a boiling point between 30° C. and 400° C.

In accordance with one embodiment the substance is in the liquid state at ambient temperature and said storage container further contains an internal cellular retention member impregnated with said substance in the liquid state.

In accordance with one embodiment the cellular retention member includes a material chosen from felt, for example wool felt, and a melamine foam.

In accordance with one embodiment a plurality of cellular retaining members are in contact and disposed in the storage container.

In accordance with one embodiment the storage container has no opening other than the drain orifice and the storage container contains in addition to the substance a gas phase occupying at least 20% of the volume of the storage container.

The first temperature may be set in different ranges. If the diffusing apparatus is intended to be used in the open air the first temperature would in particular be chosen as a function of local climatic data. In accordance with embodiments the first temperature is for example between 1° C. and 50° C. inclusive or between 5° C. and 40° C. inclusive or between 10° C. and 35° C. inclusive or between 15° C. and 25° C. inclusive.

In accordance with one embodiment the substance has a viscosity varying as a function of temperature, said viscosity being such that in the position of use the substance is not able to flow through the porous body at any ambient temperature, the first temperature being higher than 0° C., and the substance flows through the porous body at a second temperature above the first temperature.

In accordance with one embodiment the substance is in the liquid state at ambient temperature. For example the substance may have at atmospheric pressure a melting point between −70° C. and 0° C. inclusive.

In accordance with one embodiment the substance is in the solid state at ambient temperature. For example the substance may have at atmospheric pressure a melting point above 30° C., for example between 30° C. and 40° C. inclusive.

In accordance with one embodiment the removable assembly includes a plurality of storage containers for containing different substances, each of the storage containers having a drain orifice communicating with the dispensing member. Such storage containers may be produced in various ways, for example with internal partitioning of a single reservoir or by means of plurality of distinct reservoirs.

The invention also proposes a diffusing apparatus intended to disperse into the air in the vapor state a substance in the liquid or solid state at ambient temperature, the diffusing apparatus including:
a removable assembly in accordance with any of the embodiments described hereinabove,
a fixed part including a diffusing module that defines a housing in which said removable assembly can be inserted, and
a heating member,
in which the heating member is arranged in the housing so as to be engaged in the central free space of the storage container and to come into contact with the contact surface on the porous body of the removable assembly when the removable assembly is inserted in the housing in the position of use.

In accordance with embodiments such diffusing apparatus may have one or more of the following features.

In accordance with one embodiment the heating member is received in part in the void in the porous body so as to come into contact with the contact surface.

In accordance with one embodiment the heating member comprises an electrical resistance in contact with the contact surface.

In accordance with one embodiment the diffusing apparatus further includes an electronic circuit card, the heating member being supplied with electrical energy by the electronic circuit card.

In accordance with one embodiment the diffusing apparatus further includes a control device configured to control the heating member as a function of a setpoint temperature defining a temperature of the porous body.

In accordance with one embodiment the control device is on the electronic circuit card.

In accordance with one embodiment the fixed part further includes an air intake and an aeration system configured to create a flow of air from the air intake to the evaporation surface of the hollow body.

In accordance with one embodiment the aeration system includes at least one fan.

In accordance with one embodiment said control device is configured to control said at least one fan.

In accordance with one embodiment the fixed part further includes an electrical power supply module. Such an electrical power supply module may include one or more batteries, a mains plug, a direct current electrical connector or a combination of the above.

In accordance with one embodiment the diffusing module can be fixed to the electrical power supply module.

In accordance with one embodiment the air intake includes a gap between the electrical power supply module and the diffusing module.

### BRIEF DESCRIPTION OF THE FIGURES

The invention will be better understood and other aims, details, features and advantages thereof will become more clearly apparent in the course of the following description of particular embodiments of the invention given by way of non-limiting illustration only and with reference to the appended drawings.

FIG. 1 is a general view in perspective of a diffusing apparatus in accordance with a first embodiment.

FIG. 2 is a view in section of the diffusing apparatus from FIG. 1.

FIG. 3A is a view in section of a storage container of a removable assembly intended to be received in a housing of the diffusing apparatus from FIGS. 1 and 2.

FIG. 3B is a view from above of the storage container from FIG. 3A.

FIG. 3C is a view from above analogous to FIG. 3B showing a variant of the storage container.

FIG. 4 is a view in section of the storage container from FIG. 3A combined with a dispensing member.

FIG. 5 is a view of the detail V from FIG. 4 to a larger scale and partly in section and partly in perspective.

FIG. 6A is a perspective view of the storage container and the dispensing member from FIGS. 4 and 5 together with a support.

FIG. 6B is a perspective view in the direction of the arrow VIB in FIG. 6A.

FIG. 7 is a perspective view analogous to FIG. 6A additionally showing a tamper-evident indicator in place between the storage container and the support.

FIG. 8 is a partial view in perspective of the casing of the dispensing apparatus from FIG. 1 as seen from above in the direction of the arrow VIII in FIG. 1.

FIG. 9 is a partial perspective view of a diffusing apparatus in accordance with a second variant embodiment.

FIG. 10 is a partial perspective view of a diffusing apparatus in accordance with a third variant embodiment.

FIG. 11 is a partial perspective view of a diffusing apparatus in accordance with a fourth variant embodiment.

FIG. 12 is a partial perspective view of a diffusing apparatus in accordance with a fifth variant embodiment.

FIG. 13 is a view in section of a variant of the removable assembly represented in FIGS. 4 and 5.

FIG. 14 is an exploded view of the storage container of the removable assembly from FIG. 13.

FIG. 15A is a side view of a storage container.

FIG. 15B is a view in section of the storage container from FIG. 15A.

FIG. 15C is a side view of a wick support element.

FIG. 16A is a side view of another variant of the removable assembly represented in FIGS. 4 and 5 including the storage container represented in FIG. 15A and FIG. 15B, the wick support element from FIG. 15C and a dispensing member.

FIG. 16B is a view in section of the removable assembly from FIG. 16A.

FIG. 17 is a view in section analogous to FIG. 2 showing a diffusing apparatus conforming to a sixth variant embodiment.

FIG. 18 is a view in section analogous to FIG. 16B showing another variant of the removable assembly represented in FIGS. 4 and 5.

### DESCRIPTION OF EMBODIMENTS

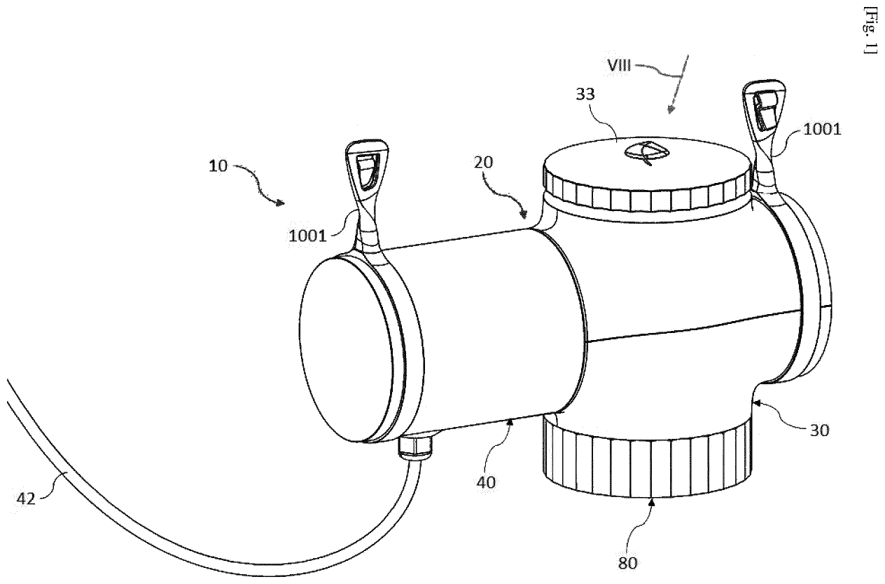

FIG. 1 is a perspective view of a first variant of a diffusing apparatus intended to disperse into the air in the vapor state a substance in the liquid state at ambient temperature. This diffusing apparatus bears the reference 10 in the drawings; for convenience it will be called hereinafter “the apparatus 10”.

The apparatus 10 includes a fixed part generally designated by the reference 20.

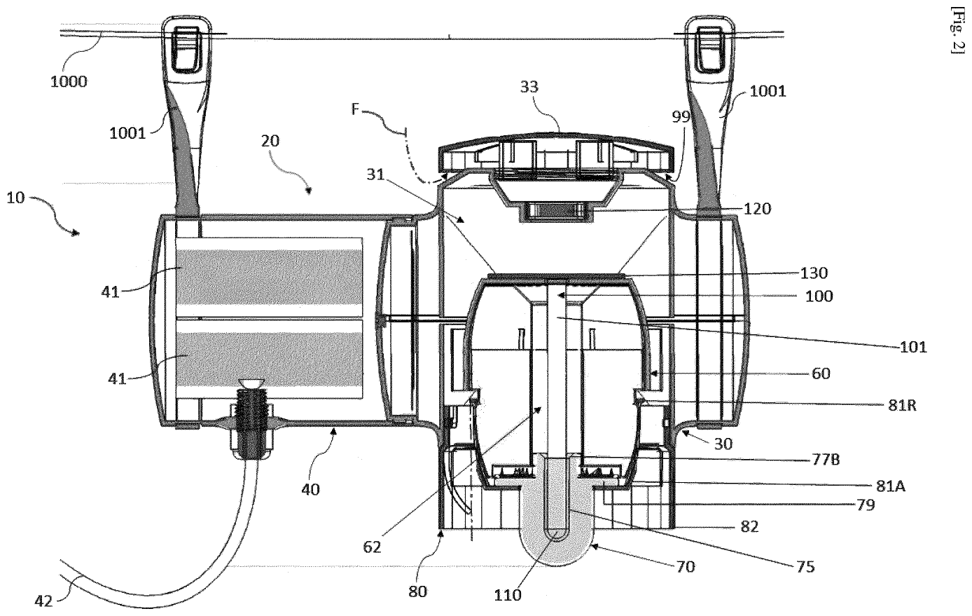

The fixed part 20 is described first of all. The fixed part 20 includes a diffusion module 30 and an electrical power supply module 40. FIG. 2 is a view in section of the apparatus 10 and therefore reveals components of the apparatus 10 disposed inside the diffusing module 30 and the electrical power supply module 40.

The diffusing module 30 defines a housing 31, here of globally cylindrical section. The housing 31 is adapted to receive a removable assembly 50 described hereinafter.

The electrical power supply module 40 includes one or more batteries 41 in order to supply the electrical components of the diffusing module 30 with electricity. A power supply cable 42 is connected to an external electrical power supply, such as a photovoltaic panel (not represented), in order to supply the apparatus 10 with electricity.

The apparatus 10 is suspended from a taut cable 1000 (not represented in FIG. 1 but represented in FIG. 2) such as a tie-in cable by means of two suspension rings 1001. The suspension rings 1001 are disposed on respective opposite sides of the diffusing module 30 as represented in FIGS. 1 and 2.

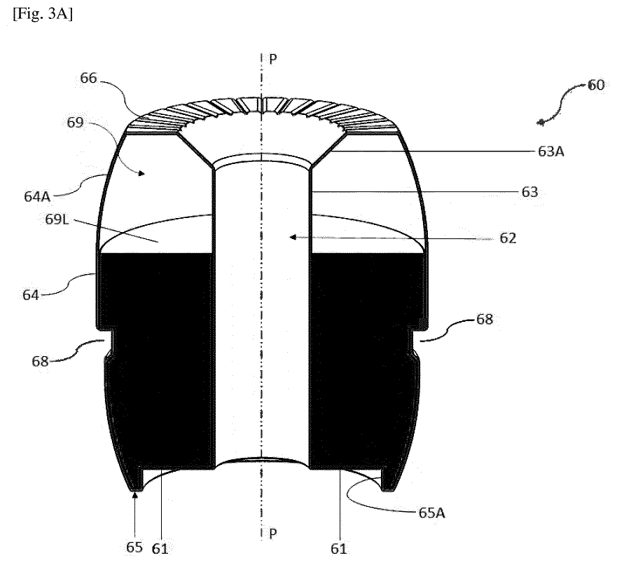

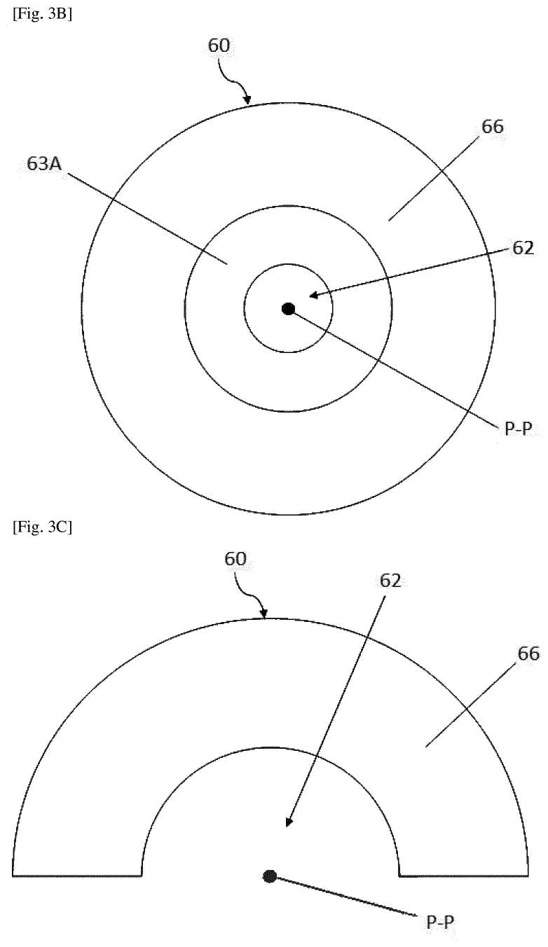

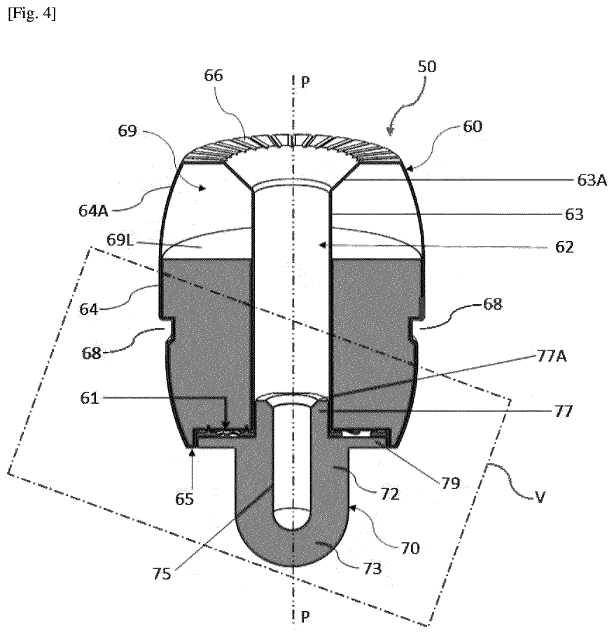

The removable assembly 50 will now be described with reference to FIGS. 3A to 7. The removable assembly 50 includes a storage container 60 and a wick 70. The wick 70 is fastened to the storage container 60 so that the removable assembly 50 can be inserted in one piece into the housing 31 when holding the removable assembly 50.

FIG. 3A is a view in section of the storage container 60 on its own. As can be seen in FIG. 3A the storage container 60 has an annular cylindrical exterior shape extending along a main axis P-P. By “annular cylinder” is meant that a section of the storage container 60 perpendicular to the axis P-P is of annular shape, as depicted schematically in FIG. 3B. The storage container 60 therefore has at its center and near the axis P-P a central free space 62. The central free space 62 is delimited by a proximal wall 63 of the storage container 60. The interior volume 69 of the storage container 60 is delimited by the proximal wall 63, a distal wall 64 farther from the axis P-P than the proximal wall 63, an upper wall 66 and a lower wall 65.

As seen better in FIG. 3A the lower wall 65 includes a spot facing 65A. The central free space 62 opens into the spot facing 65A. A drain orifice 61 is formed in all or part of the bottom of the spot facing 65A. The drain orifice 61 is oriented downwards in a position of use of the removable assembly 50, this position of use being represented in FIG. 2.

The terms “downward”, “lower” and “upper” must be understood with reference to the acceleration due to gravity in the position of use.

The storage container 60 may exhibit symmetry of revolution about the axis P-P.

In one embodiment the storage container 60 is made by injection molding an appropriate plastic material such as polypropylene (PP) for example. One or more pressurization vents (not represented) may be formed in the upper wall 66 and/or the distal wall 64 in order to guarantee that the liquid substance contained in the storage container 60 is able to continue to flow until the storage container 60 is emptied.

Alternatively, the storage container 60 includes no opening other than the drain orifice 61 and contains, in addition to the liquid substance, a gas phase occupying at least 20% of the volume of the storage container 60.

Near the upper end of the storage container 60 the proximal wall 63 may have an inclined portion 63A so that the section of the central free space 62 increases toward the upper end of the storage container 60. The distal wall 64 may also have an inclined portion 64A so that the distal wall 64 comes closer to the axis P-P in the direction toward the upper end of the storage container 60.

In another embodiment the walls of the storage container 60, with the possible exception of some or all of the lower walls 65, may be flexible so that the interior volume 69 of the storage container 60 decreases because of the effect of atmospheric pressure as the storage container 60 is emptied. Techniques for producing such flexible walls are known as such and are not described in detail here.

In further embodiments the section of the storage container 60 perpendicular to the axis P-P is not necessarily of annular shape. Any exterior shape of the storage container 60 may be envisaged provided that this exterior shape is concave and at least partially surrounds a central free space 62 and provided that the heating member 100 can be received in the central free space 62 as described below. In particular, the storage container 60 may have a concave exterior shape in the shape of a “C” as represented diagrammatically in the view from above in FIG. 3C or a more complex concave exterior shape.

The interior volume 69 of the storage container 60 may optionally be at least partly filled with a polymer foam (not represented) impregnated with the liquid substance.

Alternatively, the interior volume 69 may simply be filled with the liquid substance. In the drawings the reference 69L indicates a free surface of the liquid substance.

In some variants that are not represented the interior volume 69 may contain a plurality of different liquid substances. To this end the interior volume 69 may be partitioned so as to define a plurality of interior sub-volumes, each interior volume 69 containing a liquid substance and having a drain orifice 61 for that liquid substance. In accordance with other variants that are not represented the removable assembly 50 may comprise a plurality of storage containers 60, which may be identical, each containing a liquid substance and all connected to the wick 70 to be described now. These variants make it possible to provide a removable assembly 50 containing a plurality of liquid substances that are not mixed until the moment at which they are diffused by the apparatus 10.

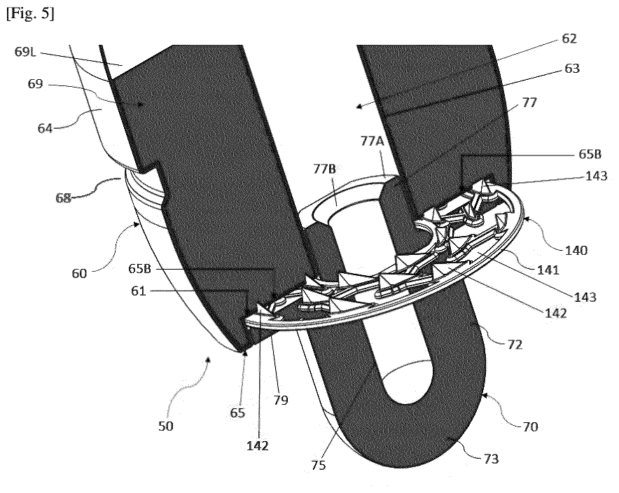

The storage container 60 together with a wick 70 have been represented in section in FIGS. 4 and 5.

The wick 70 is positioned at the outlet of the drain orifice 61 so that the liquid substance is able to pass from the interior space 69 of the storage container 60 to the wick 70 and thence to evaporation surfaces consisting of the exterior walls of the wick 70. Here the wick 70 includes a cylindrical central portion 72 and a projecting portion 73 that here is hemispherical but which could equally well be in the form of a spherical dome. Furthermore, a void 75 extends in the wick 70, here parallel to the axis P-P and therefore in line with the central free space 62. The void 75 is preferably a blind void as represented in the figures. The void 75 may or may not extend in the wick 70 as far as the projecting portion 73. Alternatively, the void 75 is not blind, that is to say discharges through the projecting portion 73.

The wick 70 may exhibit symmetry of revolution about the axis P-P.

The wick 70 may be mounted on the storage container 60 in various ways. In the example represented in FIGS. 4 and 5 the wick 70 includes a cylindrical spigot 77 so as to close off the lower end of the central free space 62. The spigot 77 is for example a force fit in the central free space 62. The upper surface of the spigot 77 bears the reference 77A in the figures.

The wick 70 is made partially or entirely of porous material, for example of wood, textile, ceramic or polymer. Another example of a possible porous material is a porous metal obtained by sintering a metal powder or a metal alloy powder. Such porous metals are known as such and the techniques for obtaining them are not described in detail here.

As already mentioned hereinabove the storage container 60 includes a drain orifice 61 that is oriented downwards in a position of use of the removable assembly 50. The wick 70 is positioned at the outlet of this drain orifice 61 so as to receive and, because of its porosity, to be impregnated with the liquid substance contained in the interior volume 69 of the storage container 60.

FIG. 5 is a view to a larger scale partially in perspective and partially in section of the detail V from FIG. 4 and therefore provides a better view of the connection between the wick 70 and the drain orifice 61. In the embodiment represented the wick 70 includes a flange 79 facing the drain orifice 61. The drain orifice 61 is blocked by a foil 65B. The removable assembly 50 further includes a perforator device 140 between the flange 79 and the drain orifice 61. The perforator device 140 includes an annular main body 141 the dimensions of which are such as to allow the spigot 77 of the wick 70 to pass through it as its center. The main body 141 includes a plurality of teeth 142 and a plurality of gaps 143 between the teeth 142. The teeth 142 are disposed so as to be able to perforate the foil 65B. Once the foil 65B has been perforated by the teeth 142 the liquid substance flows through the holes made by the teeth 142 and then through the gaps 143 and wets the flange 79 and then the rest of the wick 70 by capillarity.

The perforator device 140 may for example be made by injection molding an appropriate polymer. Alternatively, it could also be a metal part, for example a casting, or a ceramic part. Furthermore, the perforator device 140 could consist of a plurality of annular sectors, possibly but not necessarily fastened to one another.

The teeth 142 and the gaps 143 are preferably regularly spaced so as to favor uniform impregnation of the wick 70 by the liquid substance.

The geometry of the teeth 142 and the gaps 143 represented in FIG. 5 is no more than an example. Numerous other configurations are possible. Furthermore, in addition to or instead of the teeth 142 hollow or solid teeth may be disposed on the perforator device 140 so as to perforate the foil 65B as just described.

The connection that has just been described between the wick 70 and the interior volume 69 of the storage container 60 via the drain orifice 61 is no more than an example. Numerous other connections are possible. In particular, if the interior volume 69 of the storage container 60 is at least in part filled with a polymer foam impregnated with the liquid substance the wick 70 may include one or more spigots that pass through the drain orifice 61 and come into contact with this polymer foam. One or more seals (not represented) can then be disposed in such a manner as to guarantee a sealed connection between the wick 70 and the drain orifice 61.

Returning to FIG. 2, the removable assembly 50 is seen in its position of use in the housing 31 defined by the diffusing module 30. A heating member generally designated by the reference 100 is disposed in the diffusing module 30. The heating member 100 includes a rod 101 carrying at its lower end an electrical resistance 110. Because of the presence of the central free space 62 the rod 101 is able to extend through the central free space 62 so that the electrical resistance 110 comes into contact with the walls of the void 75 when the removable assembly 50 is in place in the housing 31. By supplying the electrical resistance 110 with electricity or not, the flow of the liquid substance through the wick 70 can be controlled in accordance with the physical principles described in the documents WO 2019/243734 A1 and WO 2020/254733 A1.

The upper surface 77A may optionally be connected to the void 75 by an inclined spigot 77B so as to facilitate insertion of the rod 101 and the electrical resistance 110 in the void 75.

The heating member 100 may include an electronic circuit card 130 for the supply of electricity to and control of the electrical resistance 110. For example a control device (not represented) such as a microprocessor can be placed on the electronic circuit card 130 in order to control the electrical resistance 110 as a function of a setpoint temperature of the wick 70. This setpoint temperature may be measured by a temperature sensor (not represented) carried for example by the rod 101. The electronic circuit card 130 may be disposed at various locations in the apparatus 10. Purely by way of example, in FIG. 2 the electronic circuit card 130 is represented as being disposed in the housing 31 above the removable assembly 50.

The apparatus 10 further includes an aeration system configured to create a flow of air from an air intake 99 toward the evaporation surfaces consisting of the exterior walls of the wick 70. This aeration system includes a fan 120. In the example represented the fan 120 is disposed above the removable assembly 50. The fan 120 takes the flow of air from the air intake 99 to the wick 70. The air intake 99 is advantageously a gap between the diffusing module 30 and a cover 33 fastened to the diffusing module 30. The chain-dotted line arrow F in FIG. 1 depicts the direction of the entry of air via this gap.

An air filter (not represented) may advantageously be disposed between the air intake 99 and the fan 120 in order to limit the risk of the wick 70 being soiled by undesirable particles from outside.

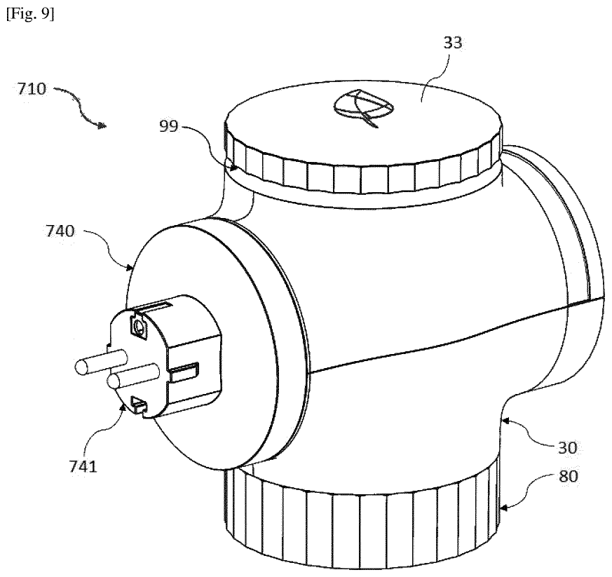

Furthermore, as can be seen in FIG. 8 the diffusing module 30 may include fins 309 between the interior wall of the diffusing module 30 and a casing 39 intended to receive the storage container 60. The fins 309 may be conformed in such a manner as to guide the flow of air toward the wick 70. It is further seen in FIG. 8 that the bottom of the casing 39 may be provided with a bottom plate 39F to support the electronic circuit card 130. A through-hole 39FF is then made in the bottom plate 39F to allow the rod 101 and the electrical resistance 110 to pass through it.

The fan 120 may be controlled by the control device mentioned hereinabove carried by the electronic circuit card 130.

The removable assembly 50 may be held in place in the housing 31 in various ways provided that the removable assembly 50 can be removed from the housing 31 in order to be replaced identically when the liquid substance contained in the storage container 60 has all been used. For example the removable assembly 50 may be screwed or clipped into the housing 31.

A particular variant of the removable assembly 50 is described hereinafter with reference to FIGS. 2, 6A, 6B and 7 that facilitates placing it in the housing 31. As represented in these figures the removable assembly 50 includes a support 80 in addition to the storage container 60 and the wick 70.

The support 80 includes a central body 81 and a peripheral body 82.

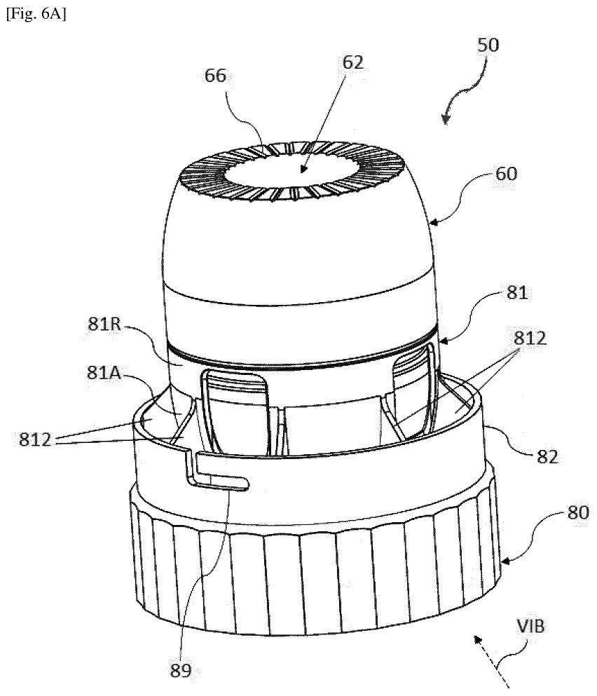

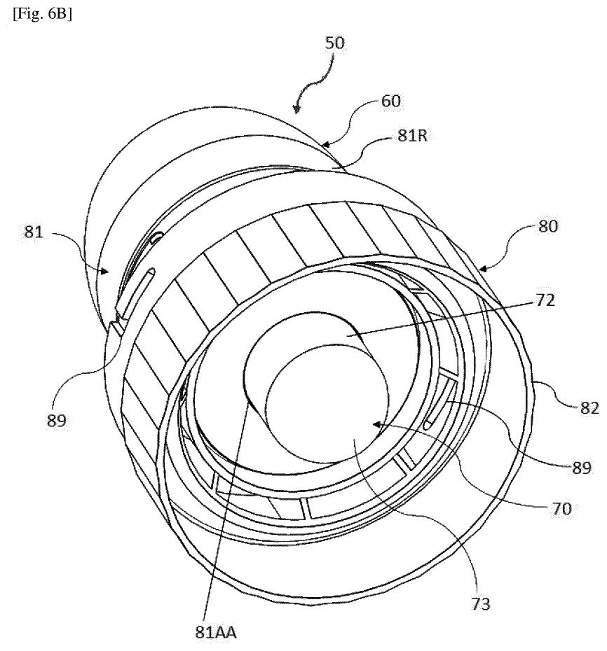

The central body 81 includes at least one central portion 81A (cf. FIG. 6A) which is tapered so as to have bear on it a lower portion of the storage container 60 and the flange 79 of the wick 70 (cf. FIG. 2). Furthermore, the central body 81 includes a through-orifice 81AA the dimensions of which are chosen to allow the wick 70 to pass through it, as can be seen in FIGS. 2 and 6B in particular. To be more precise the through-orifice 81AA allows at least the projecting portion 73 and possibly a part of the central portion 72 to pass through it, as can be seen in FIG. 6B.

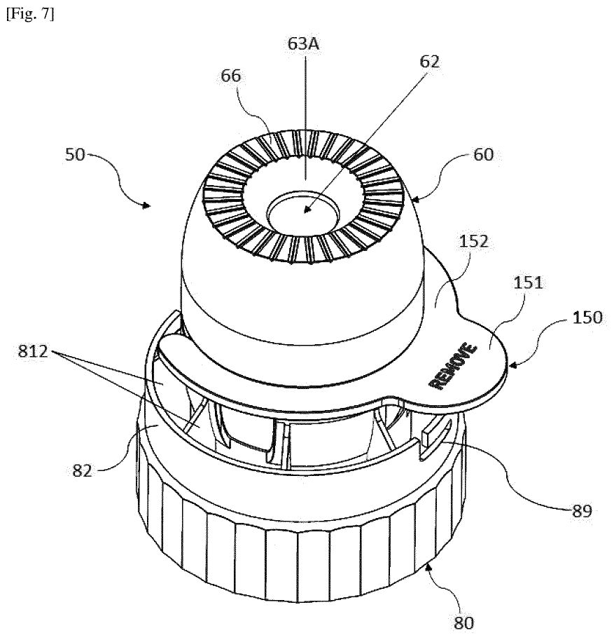

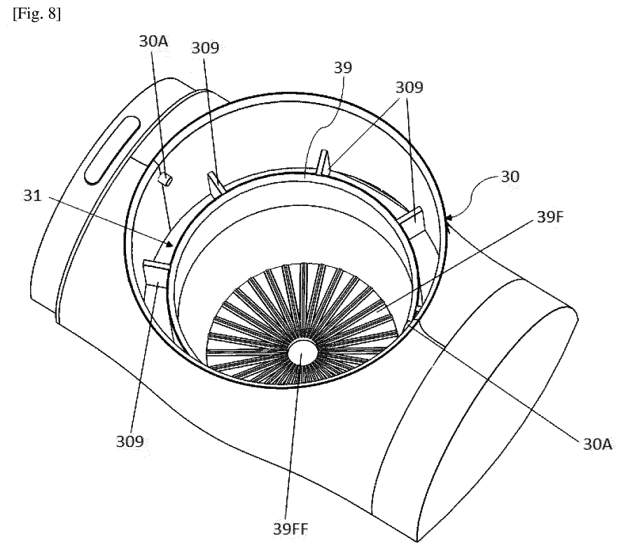

The peripheral body 82 is annular and fastened to the central body 81. The peripheral body 82 is intended to be fixed to the interior wall of the diffusing module 30. For example the peripheral body 82 is fixed to the interior wall of the diffusing module 30 by means of a bayonet coupling, here including notches 89 (FIGS. 6A and 7) that are carried by the peripheral body 82 and cooperate with corresponding pins 30A (FIG. 8) that are carried by the interior wall of the diffusing module 30. Here the peripheral body 82 is fastened to the central body 81 by being formed in one piece with ribs 812 disposed between the central body 81 and the peripheral body 82. The ribs 812 may be at least in part conformed in such a manner as to guide the flow of air toward the wick 70.

The central body 81 could have the storage container bear on it only at the level of its tapered central portion 81A. However, the central body 81 alternatively further includes an annular bearing band 81R that is received in a groove 68 on the storage container 60.

The groove 68 and the bearing band 81R may optionally enable the fitting of a tamper-evident indicator 150 that is represented in FIG. 7. As represented, the tamper-evident indicator 150 has at least one portion 152 in the shape of a “C” that is also received in the groove 68, to be more precise above the bearing band 81R. The tamper-evident indicator 150 may also include a tongue 151 that can be gripped to facilitate removal of the tamper-evident indicator 150 by a user.

Clearly as long as the tamper-evident indicator 150 remains in place as represented in FIG. 7 the portion 152 prevents the bearing band 81R from abutting against the upper wall of the groove 68. Conversely, when the tamper-evident indicator 150 is removed, for example by pulling on the tongue 151, the support 80 can move in the direction of the upper wall 66 of the storage container 60 until the bearing band 81R abuts against the upper wall of the grove 68. As will become clearer on comparing FIGS. 2 and 5, this movement of the support 80 can in particular bring about the perforation described hereinabove of the foil 65B by the teeth 142 of the perforator device 140. It is clear that the absence of the tamper-evident indicator 150 then indicates that the foil 65B has potentially been perforated and therefore that the storage container 60 has begun to release the liquid substance.

The construction of the apparatus 10 represented in FIGS. 1 to 8 is no more than an example. Numerous other constructions can be envisaged.

A second variant of the apparatus 710 is represented partially in perspective in FIG. 9. In this figure elements identical to those described above with reference to FIGS. 2 to 8 bear identical references and are not described in detail again. The apparatus 710 differs from the apparatus 10 in that the electrical power supply module 740 includes, on the side opposite the diffusing module 30, a mains plug 741. The apparatus 710 can therefore be supplied with electricity by the mains, which in particular enables use in a closed place such as a greenhouse or a building for example.

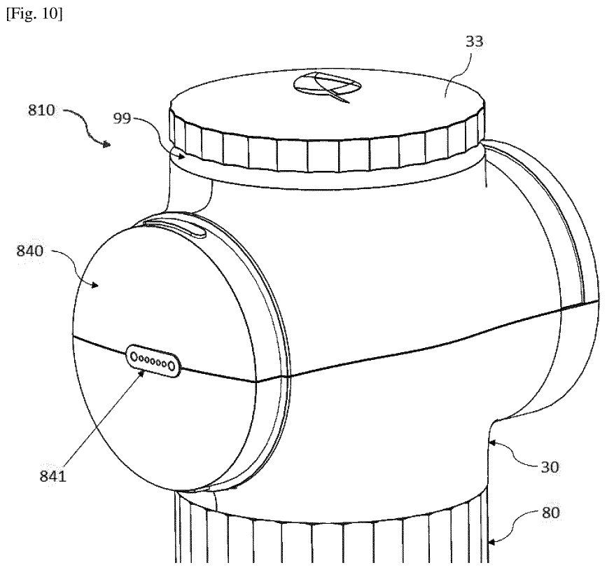

A third variant of the apparatus 810 is represented partially in perspective in FIG. 10. In this figure elements identical to those described above with reference to FIGS. 2 to 8 bear identical references and are not described in detail again. The apparatus 810 is distinguished from the apparatus 10 in that the electrical power supply module 840 includes on the side opposite the diffusing module 30 a direct current power plug 841, here one known as a Pogo Pin. The apparatus 810 can therefore be supplied with electricity by a direct current source such as a battery.

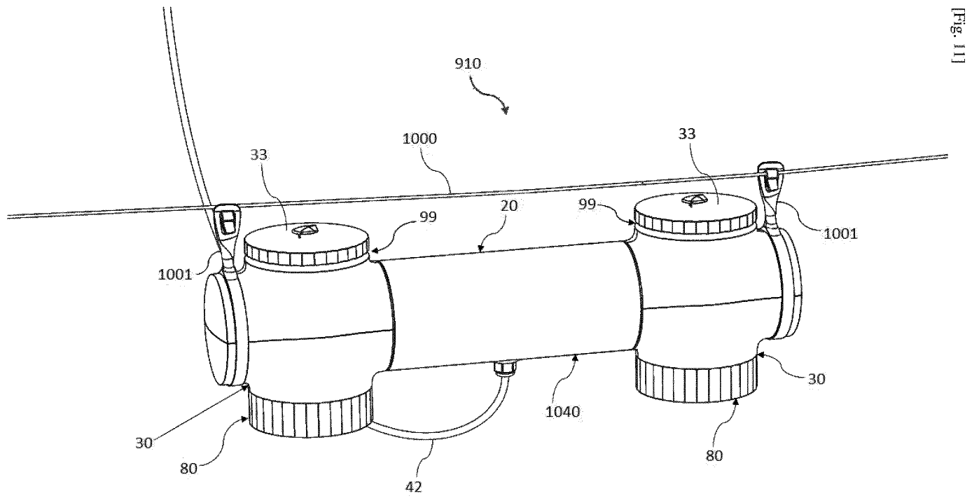

A fourth variant of the apparatus 910 is represented in perspective in FIG. 11. In this figure elements identical to those described above with reference to FIGS. 2 to 8 bear identical references and are not described in detail again. The apparatus 810 differs from the apparatus 10 in that it comprises two diffusing modules 30 disposed on respective opposite side of a common electrical power supply module 1040. The electrical power supply module 1040 may contain batteries (not represented) for example and be supplied with electricity by an electrical power supply cable 42 as described above. The apparatus 910 may be used to diffuse two different substances by inserting in the two diffusing modules 30 two removable assemblies 50 containing different substances.

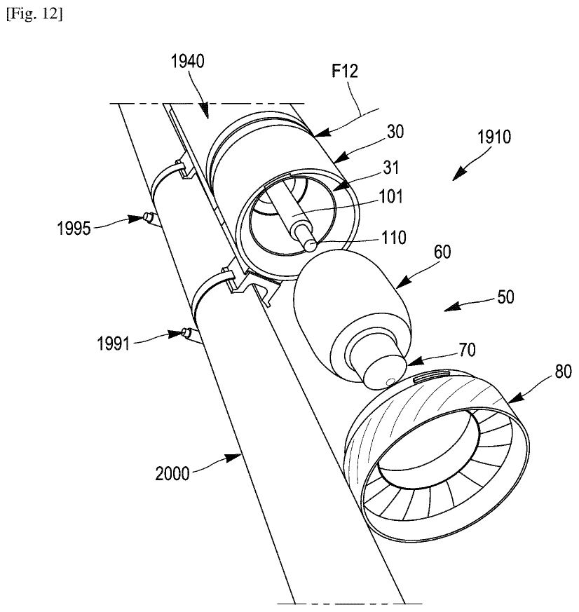

A fifth variant of the apparatus 1910 is represented in an exploded view in FIG. 12. In this figure elements identical to those described above with reference to FIGS. 2 to 8 bear identical references and are not described in detail again. The apparatus 1910 is designed to be used outdoors and at height. To this end the diffusing module 30 is fixed to a vertical post 2000, for example by means of a Jubilee clip 1991. An electrical power supply module 1940, here of cylindrical shape and the same diameter as the diffusing module 30, is disposed above the diffusing module 30. The electrical power supply module 1940 is also fixed to the post 2000, for example by means of a Jubilee clip 1995. The diffusing module 30 is fixed to the electrical power supply module 1940 by screwing it or clipping it thereto for example. The electrical power supply module 1940 may include batteries (not represented) and/or be connected to a photovoltaic panel (not represented) in order to supply the apparatus 1910 with electricity. However, the apparatus 1910 may be supplied with electricity in other ways, in particular by a mains supply. The air intake 99 is advantageously a gap (not referenced) between the diffusing module 30 and the electrical power supply module 1940. The arrow F12 in FIG. 12 depicts the direction of entry of air via this gap.

The geometry of the removable assembly 50 represented in FIGS. 2 to 7 is no more than an example. Numerous other geometries may be envisaged.

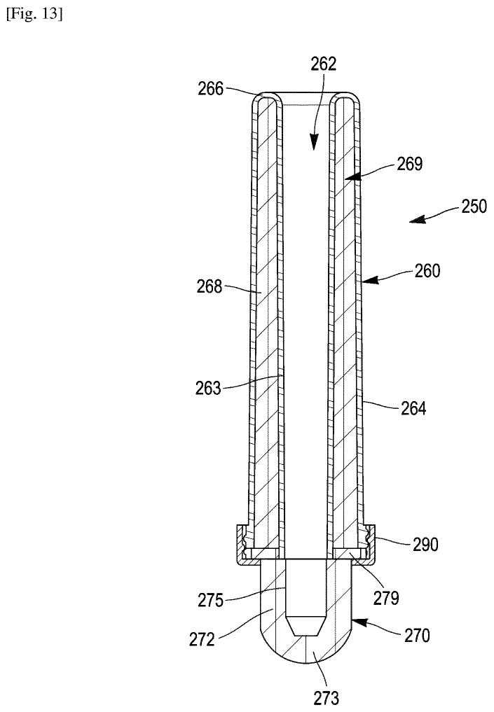

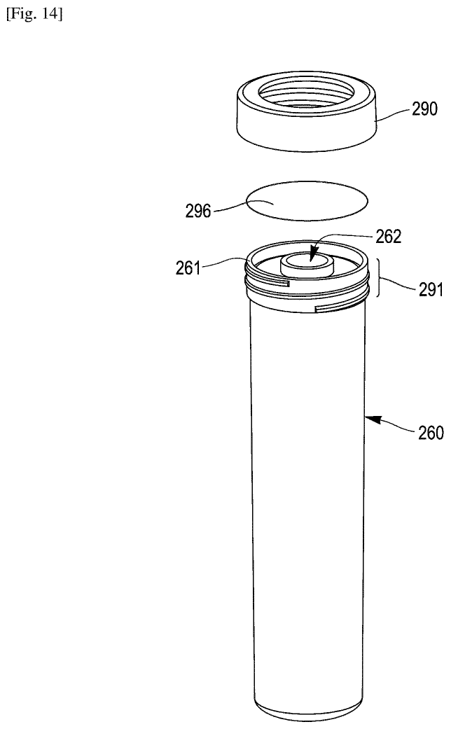

Another example of a removable assembly 250 is represented in section in FIG. 13. FIG. 14 is an exploded view of the storage container 260 of this removable assembly 250. In these figures, elements identical to those described above with reference to FIGS. 2 to 12 bear references increased by 200 and are not described in detail again unless that is necessary.

In this example the storage container 260 takes the form of a very long cylindrical part made for example by injection molding an appropriate plastic material such as polypropylene (PP) for example. The interior volume 269 of the storage container 260 is filled with a polymer foam 268 impregnated with the liquid substance. One or more pressure vents (not represented) may be formed in the vicinity of the upper end of the storage container 260, for example in its upper wall 266 and/or its distal wall 264.

Alternatively the storage container 260 has no opening other than the drain orifice 261 and contains in addition to the liquid substance a gas phase occupying at least 20% of the volume of the storage container 260.

As can be seen better in FIG. 14 the lower end of the storage container 260 carries an exterior thread 291 enabling a stopper 290 that has a corresponding interior thread (not referenced) to be screwed onto it. The stopper 290 therefore prevents loss of the liquid substance before use of the removable assembly 250, in particular during transportation thereof. The wick 270 may advantageously be retained on the storage container 260 by the stopper 290, which prevents loss of or damage to the wick 270 before use of the removable assembly 250. A foil 296 may be disposed at the lower end of the storage container 260 to block its drain orifice 261.

To install the wick 270 on the storage container 260 the stopper 290 can be unscrewed, after which the foil 296 is removed, after which the wick 270 is forced into the drain orifice 261 so that a spigot 279 carried by the wick 270 comes into contact with the polymer foam 268. In this case, as represented in FIG. 13, the stopper 290 may optionally have dimensions such that it is possible to screw the stopper 290 back onto the thread 291 after the wick 270 has been inserted in this way, which improves the seal of the junction between the wick 270 and the polymer foam 268.

In another variant that is not represented the spigot 279 may be configured so that the wick 270 can be screwed into the drain orifice 261 of the storage container 260. In this case the spigot 279 may optionally include microneedles (not represented) that perforate the foil 296 when screwing in the wick.

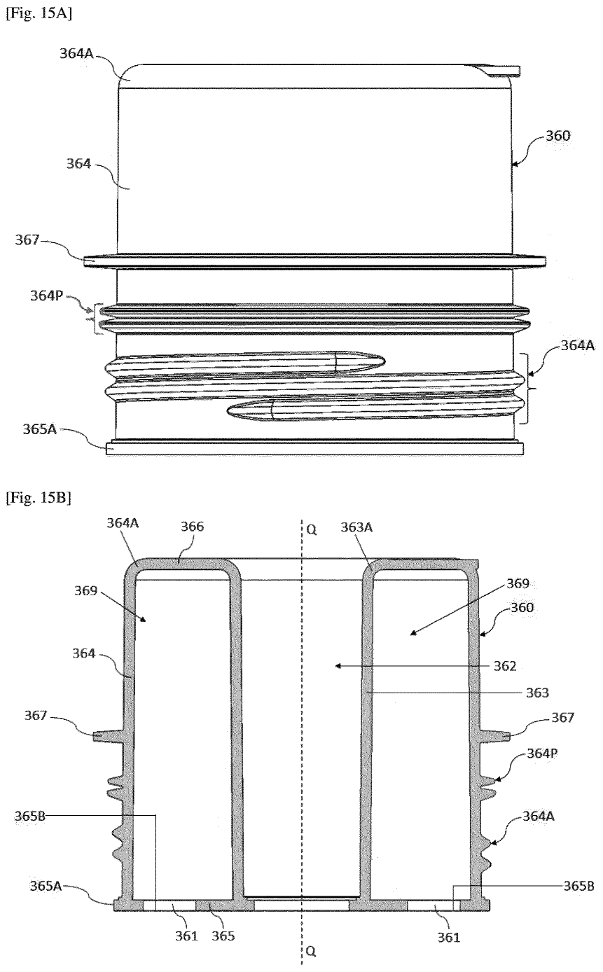

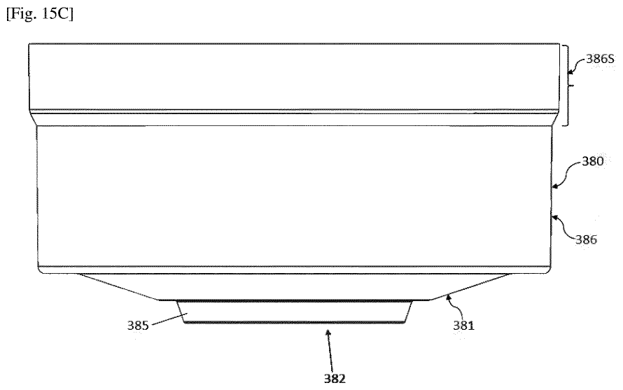

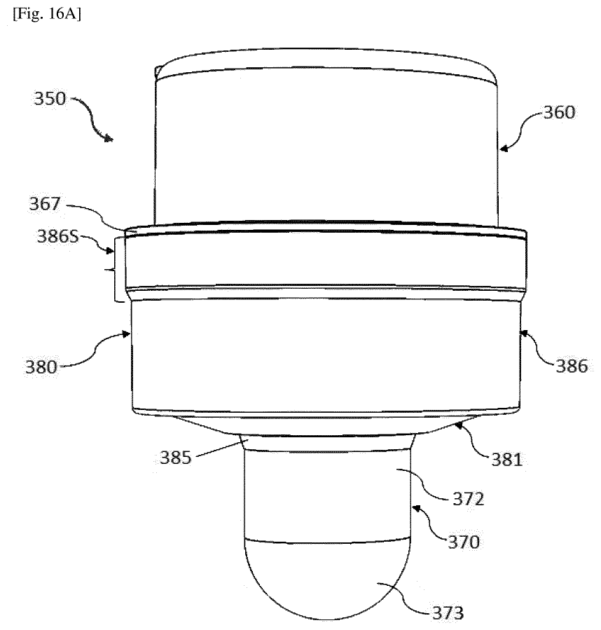

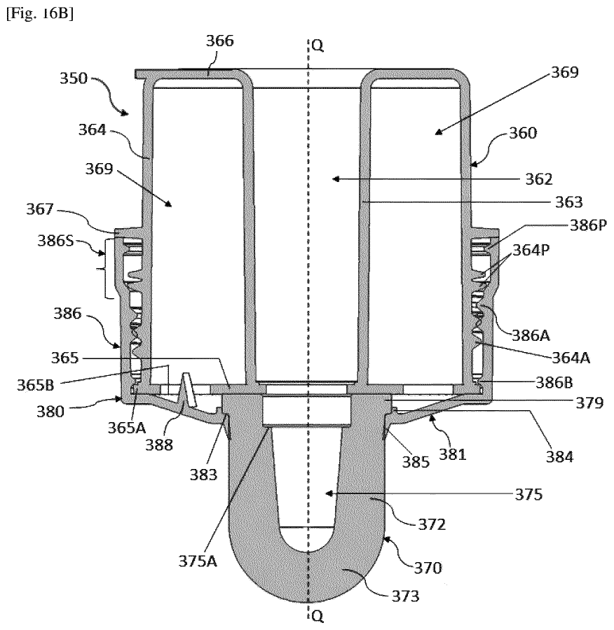

Another example of a removable assembly 350 is represented in FIGS. 15A, 15B, 15C, 16A and 16B. This removable assembly 350 includes a storage container 360, a wick 370 and a wick support element 380.

The storage container 360 is described first. FIG. 15A is a side view of the storage container 360 on its own and FIG. 15B is a view in section of the storage container 360. The storage container 360 has an annular cylindrical exterior shape extending along a main axis Q-Q; in other words, a section of the storage container 360 perpendicular to the axis Q-Q is of annular shape. The storage container 360 therefore has at its center and near the axis Q-Q a central free space 362. The central free space 362 is delimited by a proximal wall 363 of the storage container 360. The interior volume 369 of the storage container 360 is delimited by the proximal wall 363, a distal wall 364 farther from the axis Q-Q than the proximal wall 363, an upper wall 366 and a lower wall 365.

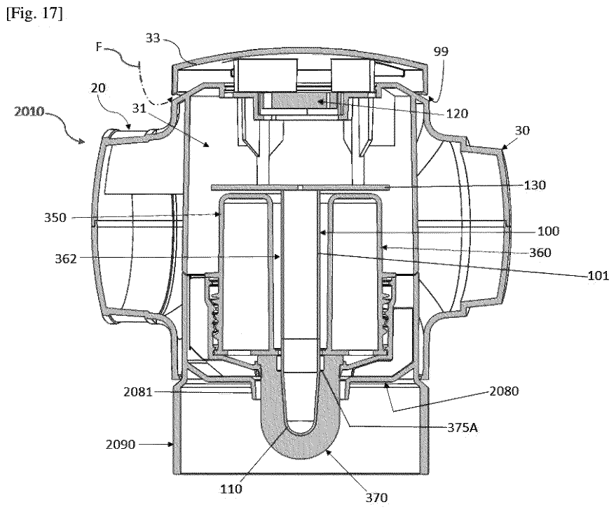

A drain orifice 361 is formed in the lower wall 365. The drain orifice 361 is oriented downwards in a position of use of the removable assembly 350 represented in FIG. 17.

The storage container 360 may exhibit symmetry of revolution about the axis Q-Q.

In accordance with one embodiment the storage container 360 is made by injection molding an appropriate plastic material such as polypropylene (PP) for example. One or more pressurization vents (not represented) may be formed in the upper wall 366 and/or the distal wall 364 in order to guarantee that the liquid substance contained in the storage container 360 is able to continue to flow until the storage container 360 is emptied. Alternatively, the storage container 360 includes no opening other than the drain orifice 361 and contains in addition to the liquid substance a gas phase occupying at least 20% of the volume of the storage container 360.

Near the upper end of the storage container 360 the proximal wall 363 may include a rounded portion 363A so that the section of the central free space 362 increases in the direction toward the upper end of the storage container 360. The distal wall 364 may additionally have a rounded portion 364A so that the distal wall 364 gets closer to the axis Q-Q in the direction toward the upper end of the storage container 360.

In another embodiment the walls of the storage container 360, with the possible exception of some or all of the lower wall 365, may be flexible so that the interior volume 369 of the storage container 360 decreases because of the effect of atmospheric pressure as the storage container 360 is emptied. Techniques for producing such flexible walls are known as such and are not described in detail here.

In further embodiments the section of the storage container 360 perpendicular to the axis P-P is not necessarily of annular shape. Any exterior shape of the storage container 360 may be envisaged provided that this exterior shape is concave and at least partially surrounds a central free space 362 and provided that the heating member 100 can be received in the central free space 362 as described below. In particular, the storage container 360 may have a concave exterior shape in the shape of a “C” or a more complex concave exterior shape.

The interior volume 369 of the storage container 360 may optionally be at least partly filled with a polymer foam (not represented) impregnated with the liquid substance. Alternatively, the interior volume 369 may simply be filled with the liquid substance.

In accordance with some variants that are not represented the interior volume 369 may contain a plurality of different liquid substances. To this end the interior volume 369 may be partitioned in such a manner as to define a plurality of interior sub-volumes, each interior volume 369 containing a liquid substance and having a drain orifice 361 for that liquid substance. In accordance with other variants that are not represented the removable assembly 350 may include a plurality of storage containers 360, which may be identical, each containing a liquid substance and all connected to the wick 370 to be described below. These variants make it possible to provide a removable assembly 350 containing a plurality of liquid substances that are not mixed until the moment they are diffused.

There are described next the wick 370, the wick support element 380 and the cooperation of the wick support element 380 with the wick 370 and the storage container 360.

The wick support element 380 is represented on its own in side view in FIG. 15C.

The wick support element 380 is configured to receive the wick 370 and to be assembled to the storage container 360 so that the wick support element 380 holds the wick 370 in position relative to the storage container 360. The removable assembly 350 can then be inserted in one piece in the housing 31 by holding the removable assembly 350. FIG. 16A is a side view of the removable assembly 350 assembled in this way and FIG. 16B is a view in section of the removable assembly 350 assembled in this way.

The wick 370 includes a cylindrical central portion 372 and a projecting portion 373 that here is hemispherical but could equally well be in the form of a spherical dome.

Furthermore, a void 375 extends in the wick 370, here parallel to the axis Q-Q and therefore in line with the central free space 362. The void 375 is preferably a blind void as represented in the figures. The void 375 may or may not extend in the wick 370 as far as the projecting portion 373. Alternatively, the void 375 is not blind, that is to say discharges through the projecting portion 373.

The wick 370 may exhibit symmetry of revolution about the axis Q-Q.

The wick support element 380 includes a support portion 381 and an assembly portion 386. The support portion 381 and the assembly portion 386 may be formed in one piece, for example by producing the wick support element 380 by injection molding an appropriate plastic material such as polypropylene (PP) for example. Alternatively the wick support element 380 could be a metal part, for example a casting, or a ceramic part.

The support portion 381 includes a central through-orifice 382 (cf. FIG. 15C). Here the orifice 382 is in line with the central free space 362. The orifice 382 has dimensions chosen to allow an upper portion of the wick 370 to pass through it as represented in FIG. 16B. To retain the wick 370 on the support portion 381 the wick 370 includes a flange 379 a lower surface of which rests on a shoulder 383 in the support portion 381 around the orifice 382. The support portion 381 may further include an internal projection 384 around the shoulder 383 to cooperate with a lateral surface of the flange 379 and/or an external projection 385 in line with the orifice 382 to cooperate with a chamfer on the wick 370 under the flange 379.

Here the support portion 381 includes a frustoconical portion to provide sufficient space for the flange 379 between the lower wall 365 of the storage container 360 and the support portion 381. The support portion 381 may exhibit circular symmetry about the axis Q-Q.

Here the assembly portion 386 has a cylindrical exterior shape extending along the axis Q-Q and of greater diameter than the storage container 360. The assembly portion 386 can therefore be assembled onto a lower part of the storage container 360.

For example the assembly portion 386 includes an interior thread 386A (cf. FIG. 16B) corresponding to an exterior thread 364A (cf. FIG. 15A, FIG. 15B and FIG. 16B) on the distal wall 364 of the storage container 360, cooperation between the interior thread 386A and the exterior thread 364A enabling the assembly portion 386 to be screwed onto the storage container 360.

The storage container 360 may include an upper flange 367 projecting from the distal wall 364 perpendicularly to the axis Q-Q to define a final position of the assembly portion 386. Additionally or alternatively the storage container 360 may include a lower flange 365A projecting from the lower wall 365 perpendicularly to the axis Q-Q to cooperate with an internal projection 386B on the assembly portion 386 and to hold the assembly portion 386 in the final position of the assembly portion 386.

In the embodiment represented the drain orifice 361 is blocked by a foil 365B. The assembly portion 386 includes a plurality of teeth 388 only one of which is represented in FIG. 16B. The teeth 388 are configured to be able to perforate the foil 365B. To be more precise the teeth 388 perforate the foil 365B when screwing the assembly portion 386 onto the storage container 360. Once the foil 365B has been perforated by the teeth 388 the liquid substance flows through the holes made by the teeth 388 and moistens the flange 379 and then the rest of the wick 370 by capillarity. It is clear that the support portion 381 and the flange 379 must have dimensions such that the liquid substance is not able to flow other than through the wick 370 as just described. A seal (not represented) can also be inserted between the support portion 381 and the flange 379.

The teeth 388 are preferably regularly spaced to favor uniform impregnation of the wick 370 by the liquid substance.

Furthermore, additionally to or instead of the teeth 388 hollow or solid needles may be disposed on the assembly portion 386 to perforate the foil 365B as just described.

Still with reference to FIGS. 15A to 16B, the storage container 360 may advantageously include in addition to the exterior thread 364A two exterior projections 364P (cf. FIG. 15A, FIG. 15B and FIG. 16B) projecting from the distal wall 364 perpendicularly to the axis Q-Q. The assembly portion 386 includes an interior projection 386P (cf. FIG. 16B). The assembly portion 386 can then be partially screwed onto the storage container 360 by means of the threads 364A and 386A until the interior projection 386P cooperates with the exterior projections 364P. The assembly portion 386 is then in an intermediate position in which the teeth 388 or needles have not yet perforated the foil 365B. The removable assembly 350 can then be transported in a pre-assembled state in which the wick support element 380 receives the wick 370 and is retained on the storage container 360 by screwing it thereto and the foil 385B has not been perforated.

Thereafter, when it is wished to use the removable assembly 350, screwing on the assembly portion 386 is completed by applying a sufficient tightening force for the interior projection 386P to be forced past the exterior projections 364P. It is clear that the tightening force to be applied for the interior projection 386P to be able to get past the exterior projections 364P is greater than the tightening force needed to screw on the assembly portion 386 as far as the intermediate position. The teeth 388 or needles therefore perforate the foil 365B and the assembly portion 386 arrives at its final position represented in FIG. 16B. To facilitate the internal projection 386P getting past the external projection 364P the upper portion 386S (cf. FIGS. 15C, 16A and 16B) of the assembly portion 386 that carries the internal projection 386P has an enlarged cross-section and/or some elasticity, for example as a result of manufacturing the assembly portion 380 by injection molding a suitable plastic material such as polypropylene (PP).

Alternatively, the assembly portion 386 may be assembled onto the storage container 360 in other ways, for example by snap-fitting, clipping, by means of a bayonet coupling, or by means of a guillotine-type closure. In this case assembling the assembly portion 386 also causes the described perforation of the foil 365B by the teeth 388 and/or the needles.

Referring to FIG. 17 there has been represented an apparatus 2010 in accordance with a sixth variant. In this figure elements identical to those described above with reference to FIGS. 2 to 8 bear identical references and are not described in detail again. Because of the presence of the central free space 362 the rod 101 can extend through the central free space 362 so that the electrical resistance 110 comes into contact with the walls of the void 375 when the removable assembly 350 is in place in the housing 31. By supplying the electrical resistance 110 with electricity or not it is possible to control the flow of the liquid substance through the wick 370 in accordance with the physical principles described in the documents WO 2019/243734 A1 and WO 2020/254733 A1.

The void 375 may optionally include a shoulder 375A (cf. FIG. 16B and FIG. 17) to facilitate insertion of the rod 101 and the electrical resistance 110 into the void 375.

The diffusing module 30 of the apparatus 2010 my optionally include a retaining portion 2080 for retaining the removable assembly 350 in place in the housing 31. For example, as represented in FIG. 17 the retaining portion 2080 retains the removable assembly 350 by supporting the removable assembly 350, here by means of the wick support element 380 of the latter. In this case the retaining portion 2080 includes a through-orifice 2081 to allow the wick 370 to pass through it. This through-orifice 2081 may contribute to guiding the flow of air created by the fan 120 along evaporation surfaces consisting of the exterior walls of the wick 370.

The diffusing module 30 of the apparatus 2010 may further include an annular portion 2090 around the wick 370. The annular portion 2090 is open under the wick 370 in the position of use of the removable assembly 350 represented in FIG. 17. The substance evaporating at the level of the exterior walls of the wick 370 can therefore be dispersed into the air while the annular portion 2090 tends to protect the wick 370, for example against mechanical damage (for example during transportation of the apparatus 2010) and/or against contact with undesirable particles from the outside.

The annular portion 2090 and the retaining portion 2080 are advantageously formed in one piece as represented in FIG. 17.

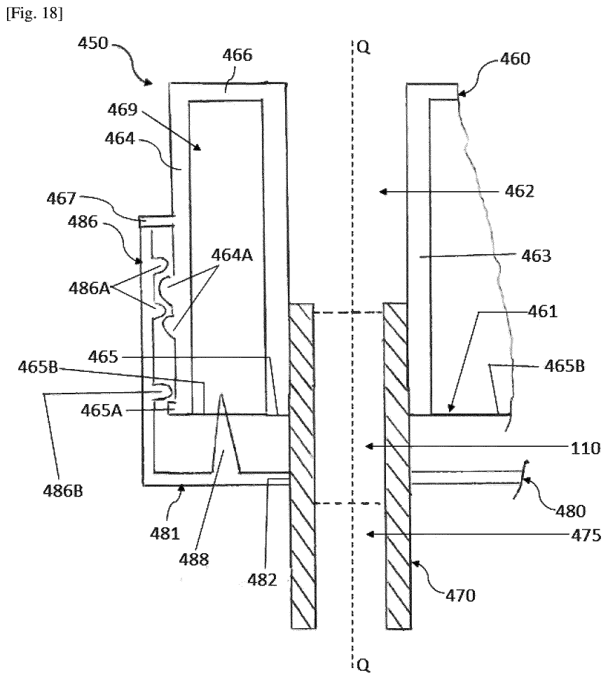

Another example of a removable assembly 450 is represented in section in FIG. 18. In this figure elements identical to those of the removable assembly 350 bear the same reference numbers increased by 100 and are not described again unless that is necessary. The removable assembly 450 differs from the removable assembly 350 in that the wick 470 has a cylindrical annular section perpendicular to the axis Q-Q and over its entire length. The wick 470 therefore includes over its entire length a cylindrical void 475 in line with the central free space 462. Part of the wick 470 is received in the central free space 462 of the storage container 460. The position of the electrical resistance 110 when the removable assembly 450 is in place in the housing 31 has been represented in dashed line in FIG. 18.

In a manner that is not represented in FIG. 18 the wick 470 may be held in place on the wick support element 481 in a manner analogous to the retention of the wick 370 on the wick support element 381 and/or be held in place in the central free space 462, for example by screwing, snap-fastening, clipping, by means of a bayonet coupling or by means of a guillotine-type closure.

There have been described until now embodiments in which the substance is in the liquid state at ambient temperature. However, the substance is alternatively in the solid state at ambient temperature. For example the substance may have at atmospheric pressure a melting point above 30° C., for example between 30° C. and 40° C. inclusive. In this case the storage container 60 (or respectively 260, 360, 460) contains the substance in the solid state at ambient temperature. The heating of the wick 70 (or respectively 270, 370, 470) by the heating member 100 causes local melting of the substance in the vicinity of the drain orifice 61 (or respectively 261, 361, 461). The substance that has become a liquid in this case is then able to flow through the wick 70 (or respectively 270, 370, 470) as described above.

Although the invention has been described in connection with particular embodiments, it is obvious that it is in no way limited to them and that it encompasses all technical equivalents and combinations of the means described if the latter fall within the scope of the invention.

The use of the verb “to include” or “to comprise” and conjugate forms thereof does not exclude the presence of elements or steps other than those stated in a claim.

In the claims, any reference sign between parentheses should not be interpreted as a limitation of the claim.

1. A removable assembly (50; 250; 350; 450) for a diffusing apparatus (10; 710; 810; 910; 1910; 2010), the diffusing apparatus for dispersing into the air in the vapor state a substance in the liquid or solid state at ambient temperature, the removable assembly comprising:
a storage container (60; 260; 360; 460) for containing the substance and including a drain orifice (61; 261; 361; 461), the drain orifice (61; 261; 361; 461) being oriented downwards when the removable assembly (50; 250; 350; 450) is in a position of use; and
a dispensing member positioned at the outlet of the drain orifice (61; 261; 361; 461) and connected to the drain orifice, the dispensing member including a porous body (70; 270; 370; 470) having an evaporation surface situated at the exterior of the storage container (60; 260; 360; 460) to evaporate the substance into the surrounding air,
wherein the storage container (60; 260; 360; 460) has a concave exterior shape surrounding at least partially a central free space (62; 262; 362; 462) so as to uncover a contact surface on the porous body (70; 270; 370; 470) on the side opposite the evaporation surface, the contact surface being accessible via the central free space (62; 262; 362; 462), and
wherein the porous body (70; 270; 370; 470) includes a void (75; 275; 375; 475) situated in line with the central free space surrounded by the storage container, and the contact surface includes an interior surface of the void (75; 275; 375; 475).

2. The removable assembly (50; 250; 350; 450) as claimed in claim 1 in which the porous body (70; 270; 370; 470) includes a cylindrical portion (72; 272; 372).

3. The removable assembly (50; 250; 350; 450) as claimed in claim 2 in which the central free space (62; 262; 362; 462) extends in a direction coaxial with the cylindrical portion (72; 272; 372) of the porous body (70; 270; 370; 470).

4. The removable assembly (50; 250; 350; 450) as claimed in claim 3 in which the void (75; 275; 375; 475) extends in an axial direction of the cylindrical portion (72; 272; 372) of the porous body (70; 270; 370; 470).

5. The removable assembly (50; 250; 350) as claimed in claim 4 in which the porous body further includes a projecting portion (73; 273; 373) of hemispherical or spherical dome shape, the projecting portion (73; 273; 373) being disposed at an axial end of the cylindrical portion (72; 272; 373) opposite the central free space (62; 262; 362).

6. The removable assembly (50; 250; 350) as claimed in claim 5 in which the void (75; 275; 375) extends into the projecting portion (73; 273; 373).

7. The removable assembly (50) as claimed in claim 1 in which the porous body (70) includes a spigot (77) engaged in the central free space.

8. The removable body (50; 250; 350; 450) as claimed in claim 1 in which said concave exterior shape of the storage container (60; 260; 360;

460) is annular.

9. The removable assembly as claimed in claim 1 in which said concave exterior shape of the storage container is in the shape of a “C”.

10. The removable assembly (50; 350; 450) as claimed in claim 1 in which the drain orifice (61; 361; 461) is blocked by a foil (65B; 365B; 465B).

11. The removable assembly (50) as claimed in claim 10 in which said removable assembly (50) further includes a perforator device (140) disposed between the porous body (70) and the foil (65B), the perforator device (140) including a plurality of teeth (142) and/or needles configured to perforate the foil (65B) when the porous body (70) is moved in the direction of the foil (65B).

12. The removable assembly (350; 450) as claimed in claim 10 in which the removable assembly (350; 450) further includes a wick support element (380; 480) configured to be assembled to the storage container (360; 460), the wick support element including a support portion (381; 481), the support portion (381; 481) being configured to support the porous body (370; 470) and including a through-hole (382; 482) through which the porous body (370; 470) passes and the support portion (381; 481) including a plurality of teeth (388) and/or needles configured to perforate the foil (365B; 465B) when the wick support (380; 480) is assembled to the storage container (360; 460).

13. The removable assembly (50; 250; 350; 450) as claimed in claim 1 in which the substance is a liquid substance that has a viscosity varying as a function of temperature, said viscosity being such that in the position of use the substance is not able to flow through the porous body (70; 270; 370; 470) at any ambient temperature below a first temperature, the first temperature being above 0° C., and the substance flows through the porous body (70; 270; 370; 470) at a second temperature above the first temperature.

14. The removable assembly as claimed in claim 1 including a plurality of storage containers containing different substances, each of the storage containers including a drain orifice communicating with the dispensing member.

15. A diffusing apparatus (10; 710; 810; 910; 1910; 2010) intended to disperse into the air in the vapor state a substance in the liquid or solid state at ambient temperature, the diffusing apparatus (10; 710; 810; 910; 1910; 2010) including:
a removable assembly (50; 250; 350; 450) as claimed in claim 1,
a fixed part (20) including a diffusing module (30) that defines a housing (31) in which said removable assembly (50; 250; 350) can be inserted, and
a heating member (100),
in which the heating member (100) is arranged in the housing (31) so as to be engaged in the central free space of the storage container and to come into contact with the contact surface on the porous body (70; 270; 370; 470) of the removable assembly when the removable assembly (50; 250; 350; 450) is inserted in the housing (31) in the position of use, and
in which the heating member (100) is received in part in the void (75; 275; 375; 475) in the porous body (70; 270; 370; 470) so as to come into contact with the contact surface.

16. The diffusing apparatus (10; 710; 810; 910; 1910; 2010) as claimed in claim 15 which further includes an electronic circuit card (130), the heating member (100) being supplied with electrical energy by the electronic circuit card (130).

17. The diffusing apparatus (10; 710; 810; 910; 1910; 2010) as claimed in claim 16 further including a control device configured to control the heating member (100) as a function of a setpoint temperature defining a temperature of the porous body (70; 270; 370; 470).

18. The diffusing apparatus (10; 710; 810; 910; 1910; 2010) as claimed in claim 17 in which the control device is on the electronic circuit card (130).

19. The diffusing apparatus (10; 710; 810; 910; 1910; 2010) as claimed in claim 15 in which the fixed part (20) further includes an air intake (99) and an aeration system configured to create a flow of air from the air intake (99) to the evaporation surface of the hollow body.
